(19) United States
(12) Patent Application Publication
(10) Pub. No.: US 2005/0033267 A1
(43) Pub. Date: Feb. 10, 2005

(54) Connector with connection mechanism adapted for releasable interconnection with tube

(75) Inventor: Christine Decaria, Salt Lake City, UT (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/635,288

(22) Filed: Aug. 6, 2003

(51) Int. Cl. A61M 25/16

(52) U.S. Cl. 604/533

(57) ABSTRACT

A connector is provided which includes a body with a conduit therethrough and a connecting mechanism for releasably securing a tube the first end of the connector. The body has a first end, a second end, an inner surface and an outer surface. The connecting mechanism desirably includes a compression fitting. The present invention is also provides a method of using a universal connector adapted for use with a medical device.

### BACKGROUND OF THE INVENTION

Enteral tubes for providing food and medication to a patient are well known. For example, U.S. Pat. No. 4,666,433, entitled Gastrostomy Feeding Device, invented by Parks and issued May 19, 1987; U.S. Pat. No. 4,701,163, entitled Gastrostomy Feeding Device, invented by Parks and issued Oct. 20, 1987; U.S. Pat. No. 4,798,592, entitled Gastrostomy Feeding Device, invented by Parks and issued Jan. 17, 1989; and U.S. Pat. No. 4,685,901, entitled Gastro-Jejunal Feeding Device, invented by Parks and issued Aug. 11, 1987 disclose earlier feeding tubes.

Referring to the illustrative drawing of FIG. 1A, there is shown a perspective view of an earlier enteral feeding device 20. The device 20 includes an elongated tubular member 51 formed from a stretchable elastomeric material such as silicone. FIG. 1B is an illustrative cross-sectional view of the tubular member 51 of the earlier device. The tubular member 51 defines a jejunal tube 22, a gastronomy tube 34 and a fluid line 46.

The jejunal feeding tube 22 (FIGS. 1A and 1B) includes an outlet end portion 24 (FIG. 1A) which can extend through a patient's stomach into the jejunum. The jejunal tube outlet end portion includes perforations 26 (FIG. 1A) which permit liquid food or medication to pass therethrough. The tube 22 (FIGS. 1A and 1B) is integrally connected to a jejunal tube inlet end portion 28 (FIG. 1A) which defines a jejunal inlet port 30 (FIG. 1A) having a removable plug cover 32 (FIG. 1A).

The gastrostomy tube 34 (FIG. 1A) is shorter than the jejunal tube 22 (FIGS. 1A and 1B) and includes a plurality of drainage inlets or food outlet ports such as inlet/outlet 36 (FIG. 1A). A gastrostomy tube end portion 37 (FIG. 1A) defines a gastrostomy inlet port 38 (FIG. 1A) having a plug cover 40 (FIG. 1A).

An inflatable balloon 42 is provided near the end of the gastrostomy tube 34 (FIGS. 1A and 1B) and is inflatable through a valve 44. The valve 44 is used to supply fluid to the balloon 42 through the fluid line 46 (FIGS. 1A and 1B).

Frictional contact between the elongated tubular member 51 (FIGS. 1A and 1B) and a locking ring 56 (FIG. 1A) is sufficiently great to prevent the tubular member 51 (FIGS. 1A and 1B) from moving further into the stomach. The locking ring 56 (FIG. 1A) remains in contact with a patient's abdominal wall during use. However, the frictional contact is sufficiently low to permit adjustment of placement of the tubular member 51 (FIGS. 1 and 1B) relative to a patient's abdomen.

Referring to the illustrative drawings of FIG. 2, there is shown a perspective view of an earlier device 20 in use. The inflated balloon 42 forms a gasket that seals the entrance to the stomach, and together with the locking ring 56, secures the device 20 in place.

While prior feeding tubes generally have been acceptable, there have been shortcomings with their use. In particular, for example, in order to provide food or medication to the jejunal inlet port 30 (FIG. 1A) of device 20 (FIG. 1A), a connector, such as a first connector 58 illustrated in FIG. 3 or a second connector 60 illustrated in FIG. 4, is inserted through the jejunal inlet port 30 (FIG. 1A). The inserted connector 58 (FIG. 3) or 60 (FIG. 4) is mechanically coupled to the jejunal inlet port 30 (FIG. 1A) and serves as a conduit between the jejunal tube 22 (FIGS. 1A and 1B) and an external feeding tube 62 or 64, shown in FIGS. 3 and 4 respectively. The external tube 62 or 64 is connected to a source of food such as a feeding bag (not shown).

In practice, connectors 58 or 60 such as those shown in FIGS. 3 and 4, for example, may be inserted into and removed from the jejunal inlet port 30 (FIG. 1A) or the gastrostomy inlet port 38 (FIG. 1A) numerous times during the course of use of the device 20 (FIGS. 1A and 2) which can be installed in a patient's stomach for extended periods of time. As mentioned above, the tubular member 51 (FIGS. 1A and 1B) which defines the jejunal tube inlet end 28 (FIG. 1A), and the gastrostomy tube end portion 37 (FIG. 1A) can be formed from a stretchable elastomeric material such as silicone. In order to produce an adequate mechanical coupling between the connector 58 (FIG. 3) or 60 (FIG. 4) and either the jejunal inlet port 30 (FIG. 1A) or the gastrostomy inlet port 38 (FIG. 1A), the connector is forced into place so as to produce a frictional engagement. Repeated insertions and removals of such connectors 58 (FIG. 3) or 60 (FIG. 4) can cause the jejunal inlet port 30 (FIG. 1A) or the gastrostomy inlet port 38 (FIG. 1A) to become somewhat stretched and deformed over time.

Unfortunately, as the jejunal and gastrostomy inlet ports 30, 38 (FIG. 1A) become more and more stretched in this manner, the tendency of a medical attendant responsible for coupling such a connector to the inlet ports 30, 38 often is to more forcibly push the connector into the jejunal or gastrostomy ports 30 or 38 resulting in still further stretching. Moreover, more force often must be exerted to dislodge a connector after such a forced insertion. Additionally, as the interior of the inlet ports 30, 38 becomes soiled with food oils, for example, an attendant may attempt to push a connector into the port even more forcibly in order to compensate for the slipperiness of such oils, causing further deformation of the port opening.

The problem of achieving a tight fit between a jejunal or gastrostomy inlet port 30 or 38 (FIG. 1A) and such connectors 58 (FIG. 3) or 60 (FIG. 4), for example, has been exacerbated by the fact that in the past, such connectors often have been available in a variety of shapes and sizes. This variety will be apparent from the illustrative drawings of FIGS. 3 and 4 in which the first and second connectors 58, 60 have quite different shapes. Consequently, in the past it often has been desirable to construct jejunal or gastrostomy inlet ports, that can accommodate any of a variety of such differently shaped connectors. Unfortunately, such earlier inlet ports often could not readily accommodate such a variety of differently shaped connectors without the need to forcibly insert or forcibly remove the connectors.

Even with the advent of feeding tubes incorporating ferrules, the variety of connectors which are frequently used therewith can still lead to the forcing of the connector and the feeding tube together to make a secure connection. Depending on the tube and connector being used this forcing still may not be sufficient to create a connection which retains the connector in the tube so as to avoid an unintentional and/or undesired disconnection. Further, if sufficient force is applied during the connection of the prior connectors and tubes it may be such that the components are difficult to separate when desired. Such difficulties in separation may result in displacement of the feeding tube and/or discomfort to the patient during the attempted separation or even after the connector is ultimately separated from the tube.

These problems are only further exacerbated by the fact that it may be desired to use a certain type or brand of feeding device while the feeding bag or feeding set and the connector affixed thereto may be of a different brand and thus may not fit or connect with the feeding device as well as one specifically intended for use therewith. As a result a less desirable feeding device may be necessary in order to obtain the desired connection with the connector of the selected feeding set or a less desirable feeding solution in a feeding set having a connector which is compatible with the selected feeding device may be required to be selected for compatibility purposes.

As previous connectors have been permanently attached or affixed to the corresponding tubing of a feed set, there has been a need for the ability to interchange a connector with a feed set without the need to permanently affix the connector to the set yet still obtain sufficient sealing, gasketing or the like so that leaks between the connector and the feeding set, and more specifically the tube thereof, do not occur. The present invention meets these needs.

### SUMMARY OF THE INVENTION

In response to the difficulties and problems discussed above, a connector configured for releasable interconnection with a tube and a second member has been developed. More specifically, one aspect of the present invention is directed to a connector adapted for use with a feeding set formed from flexible material. The connector generally includes a body with a conduit therethrough and connecting mechanism. The body has a first end, a second end, an inner surface and an outer surface. The connecting mechanism provides for the releasable connection of a tube to the first end of the connector.

Another aspect of the present invention relates to a universal connector adapted for use with a medical device. The adapter includes a body with a conduit therethrough and a mechanism for releasably securing a tube to the first end of the connector. The body has a first end, a second end, an inner surface and an outer surface. The outer surface of the body has at least two regions having different diameters and at least one bevel, wherein the bevel connects two regions along the surface of the body having different diameters.

Another aspect of the present invention is directed to a method of using a universal connector adapted for use with a medical device. The method generally includes providing a connector having a mechanism for releasably securing a tube to the connector, providing a tube having two ends, and inserting one end of the tube into the mechanism. More specifically, the method includes providing a connector having a body with a conduit therethrough, the body of the connector having a first end, a second end, an inner surface and an outer surface, where the outer surface has at least two regions having different diameters and at least one bevel, wherein the bevel connects two regions along the surface of the body having different diameters.

The invention will be more fully understood and further features and advantages will become apparent when reference is made to the following detailed description of exemplary embodiments of the invention and the accompanying drawings.

### BRIEF DESCRIPTION OF THE DRAWINGS

The purpose and advantages of the present invention will be apparent to those skilled in the art from the following detailed description in conjunction with the appended drawings in which:

FIG. 1A is a perspective view of an earlier feeding tube;

FIG. 1B is a cross-sectional view along line 1B-1B of FIG. 1A;

FIG. 2 is a perspective partially cutaway view of an earlier feeding tube installed in a patient;

FIGS. 3 and 4 are side elevation views of earlier connectors for insertion into end portions of a feeding tube;

FIG. 5 is a perspective view of a connector having a connecting mechanism in accordance with the present invention;

FIG. 5A is a exploded view of an alternative connector having a connecting mechanism in accordance with the present invention;

FIG. 6 is a perspective view of a ferrule;

FIG. 6A is an exploded view of a ferrule, a feeding tube, and a connector in accordance with the present invention;

FIG. 7 is a side elevational view of a connector in accordance with the invention interconnected with a second member;

FIGS. 8, 8A and 8B cross-sectional are views of alternative connectors in accordance with the present invention; and

FIG. 9 is a cross-sectional view of the ferrule of FIG. 6.

### DETAILED DESCRIPTION OF THE PREFERRED EMBODIMENTS

The following detailed description will be made in the context of a connector which is adapted for medical use. It is readily apparent, however, that the article of the present invention would also be suitable for use with other types of systems, circuits or conduits and the like and is not intended to be limited to medical devices or use in a medical field. In addition, the invention will be described in the context of its various configurations. It should be appreciated that alternative arrangements of the invention can comprise any combination of such configurations. As such, the use of a desired embodiment, a connector adapted for use with a feeding set and feeding tube, for ease in understanding and describing the invention shall not, in any manner, limit the scope of the invention.

Reference will now be made to the drawings in which the various elements of the present invention will be given numeral designations and in which the invention will be discussed so as to enable one skilled in the art to make and use the invention. It should be appreciated that each example is provided by way of explaining the invention, and not as a limitation of the invention. For example, features illustrated or described with respect to one embodiment may be used with another embodiment to yield still a further embodiment. These and other modifications and variations are within the scope and spirit of the invention.

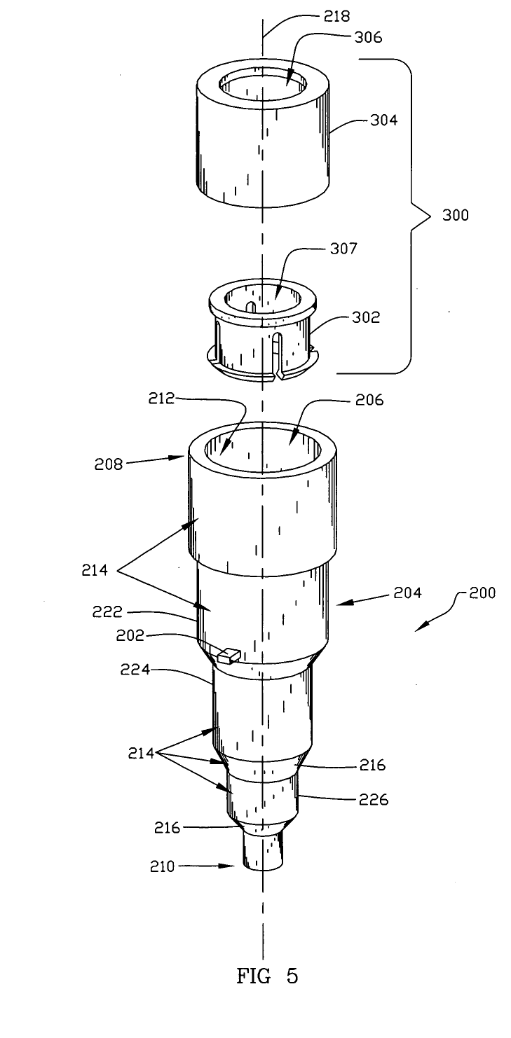

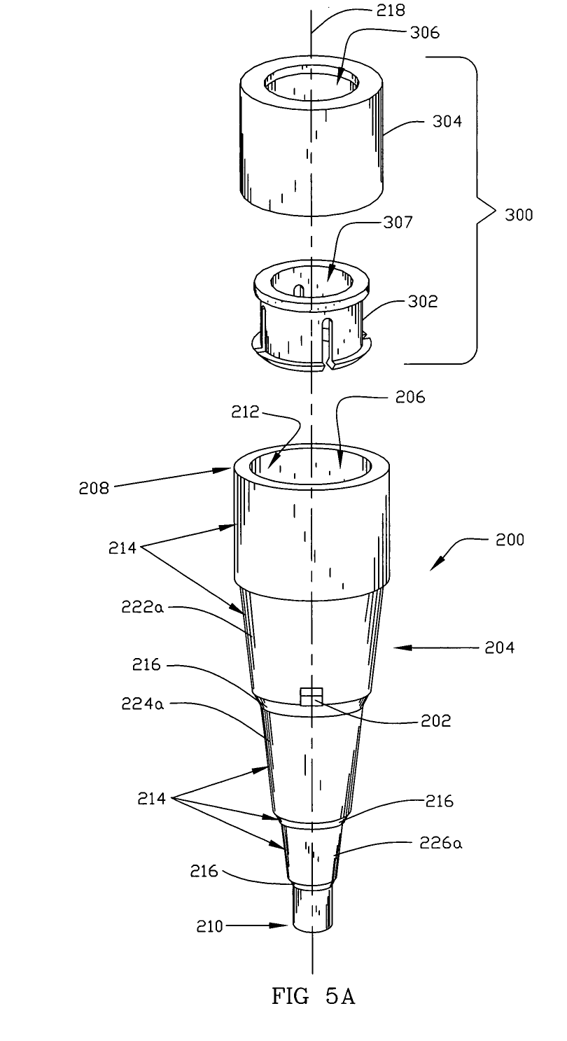

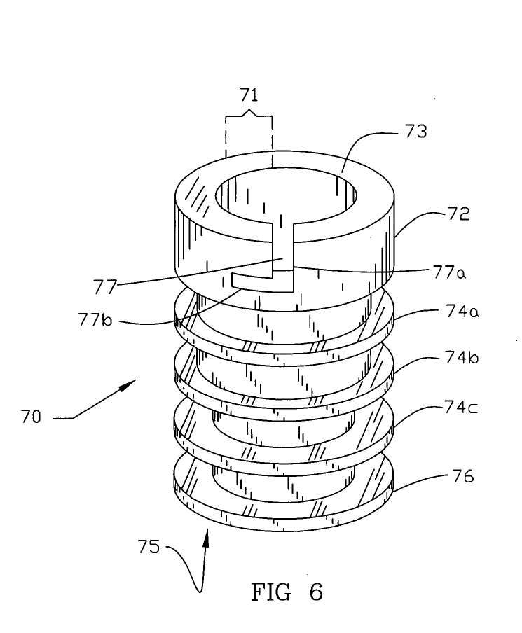

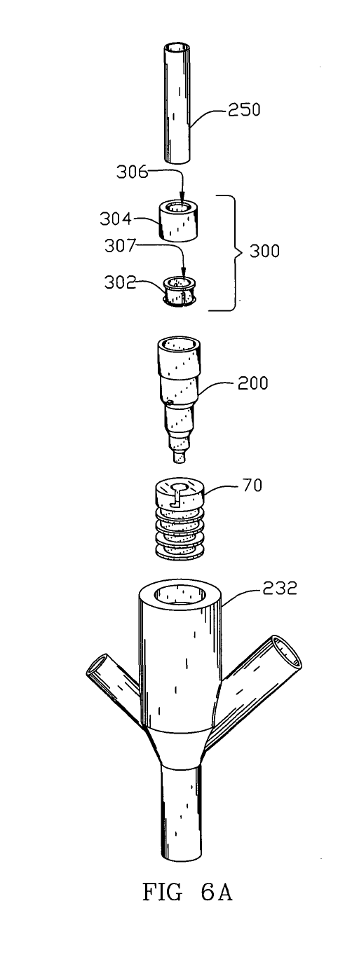

Turning now to the drawings, and FIG. 5 in particular, there is illustrated an exploded perspective view of a connector 200 in accordance with a present embodiment of the invention. The connector 200 shown includes a body 204 with a conduit or bore 206 therethrough. The body 204 has a first end 208, a second end 210, an inner surface 212 and an outer surface 214 as well as an optional protrusion 202 extending from the outer surface 214 of the body 204. Although optional, when present, the protrusion 202 is desirably configured to be received in a second member (illustrated as ferrule 70 in FIG. 6) so as to allow the connector 200 and the second member to be releasably interlocked or interconnected.

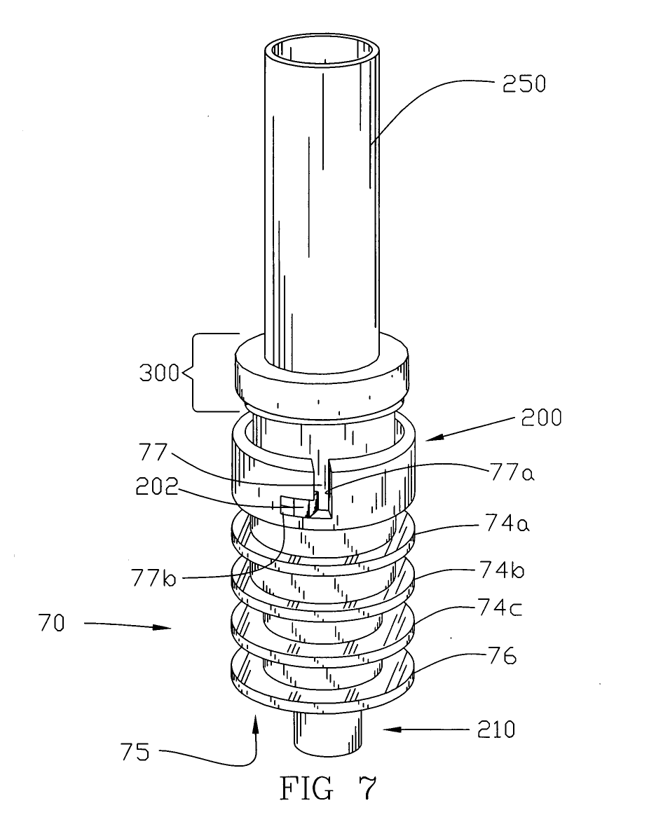

Although, as discussed in more detail herein, the protrusion may be in a variety of sizes and shapes, the protrusion 202 shown in FIGS. 5, 5A and 7 is merely exemplary and is illustrated as such for purposes of discussion and understanding. It will be appreciated that the size and shape of the protrusion 202 may be dictated in part by the second member the connector 200 is intended to be used with. A more detailed description about a suitable protrusion is described in commonly assigned copending U.S. patent application Ser. No. ______ entitled “CONNECTOR WITH PROTRUSION ADAPTED FOR INTERCONNECTION WITH SECOND MEMBER” (Attorney Docket No. 19,690A), which is herein incorporated by reference in its entirety.

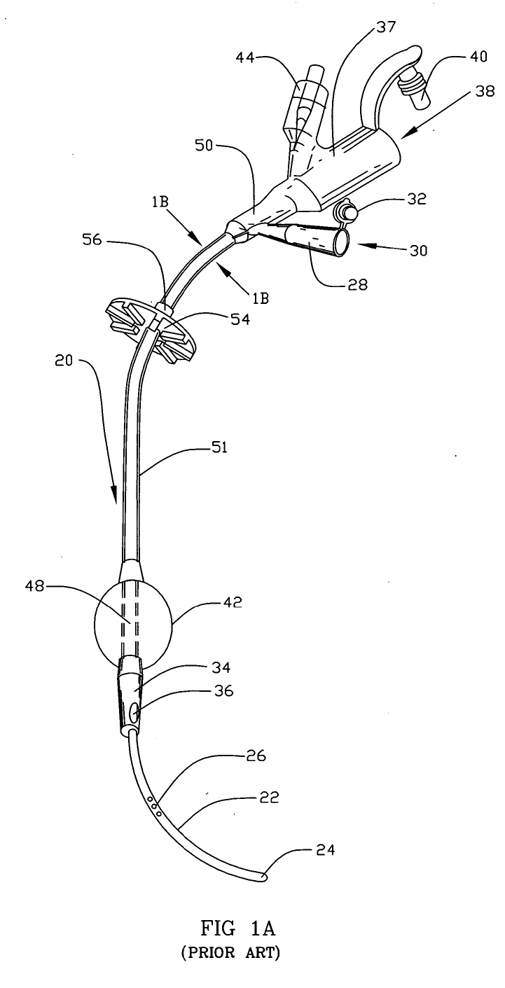

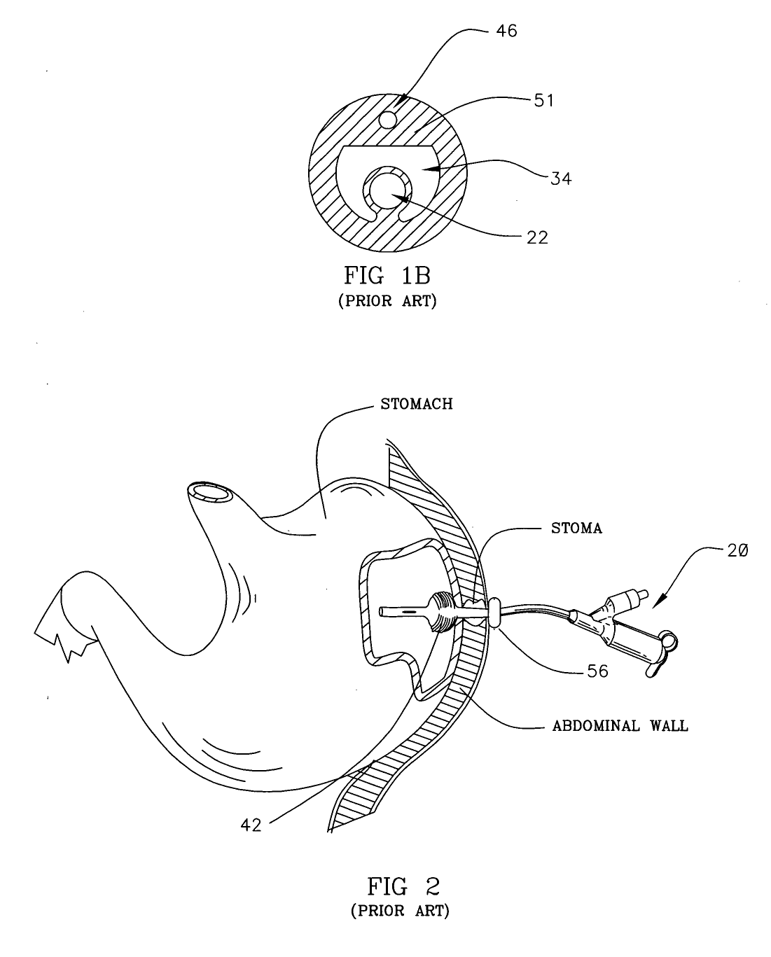

For purposes of discussion and understanding of the present invention an exemplary second member (e.g., ferrule 70) is shown in FIGS. 6 and 7. The second member shown in FIGS. 6 and 7 is merely exemplary and the dimensions of the various aspects thereof may vary in size and shape. One will appreciate that a connector 200 (FIGS. 5, 5A and 7-8B) of the present invention may be configured to be received in any suitable second member. A more detailed description of an exemplary second member is described in commonly assigned copending U.S. patent application Ser. No. ______ entitled “FERRULE AND ENTERAL TUBE INCORPORATING A FERRULE” (Attorney Docket No. 18,866), which is herein incorporated by reference in its entirety. Although the description of the present invention is made primarily in context of use with a second member, such as ferrule 70 (FIGS. 6 and 7), solely for ease of description and understanding, it will be appreciated that a connector in accordance with the present invention may used to connect to other devices with or without such a second member. That is, for example, a feeding tube such as that shown in FIG. 1 (without a ferrule) or the feeding tube 232 in FIG. 6A (with a ferrule 70) are equally suitable for use with the present invention.

Referring again to FIG. 5, the connector 200 is shown having an outer surface 214 which has regions with different diameters. Although the connector need not have regions having different diameters, it is generally desired that a connector, such as that shown as 200 in FIG. 5, have at least two such regions 222, 224 having different diameters so as to more readily allow the connector 200 to be compatible with multiple second members. Those embodiments of the present invention which have multiple regions with different diameters along the outer surface 214 of the connector 200 also desirably include at least one luer or bevel 216 that connects two regions (e.g. 222 and 224 or 224 and 226) of the outer surface 214 of the body 204 having different diameters.

In one embodiment of the present invention the outer surface 214 of the body 204 of the connector 200 may be tapered at least in part. Depending on the embodiment this taper may be, for example, a luer or bevel (e.g. 216 in FIG. 5) or it may be another region of the outer surface 214 of the body 204 such as 222a, 224a, 226a (see FIG. 5A). That is, for example, in one embodiment of the present invention the outer surface 214 of the body 204 of the connector 200 may be generally parallel to the central axis 218 of the connector 200 as in FIG. 5 or the outer surface 214 of the connector 200 may be inclined relative to the central axis 218 so as to form a taper as shown in FIG. 5A. It will be appreciated that multiple tapers may be present on one connector. Further, it is contemplated that the interior surface 212 of the connector 200 may be tapered.

Depending on the number, type and size of tapers which are present on the connector 200, the adapter can take a variety of forms and shapes including, for example, a universal connector or a “Christmas tree” connector. As discussed in more detail herein, tapers along the outer surface 214 of the connector 200 may be configured for engagement with a second member to promote or assist in the creation of a seal, lock, gasketing effect or the like between the two components.

Referring again to the protrusion 202 of the connector 200 as seen in FIGS. 5, 5A and 7, it will be appreciated that while the protrusion may be of any suitable size and shape the position of the protrusion 202 on the connector 200 may affect the dimensions of the protrusion. Although the protrusion 202 may be located on or extend from the ends 208, 210 of the connector 200, it is generally desirable for the protrusion 202 to be located or positioned along the outer surface 214 of the body 204 of the connector 200 between the first end 208 and the second end 210 of the body 204. More desirably, the protrusion will be located closer to the first end 208 than the second end 210. Furthermore, while the protrusion 202 may protrude from the connector 200 at any suitable angle relative to the central axis 218, it is generally desirable for the protrusion to protrude from the connector at angle of about 80° to about 100°, and more desirably about 90° relative to the central axis 218. Again, as will be apparent from the discussion herein, the size, shape and location of the protrusion may depend on the connector and second member intended to be used therewith.

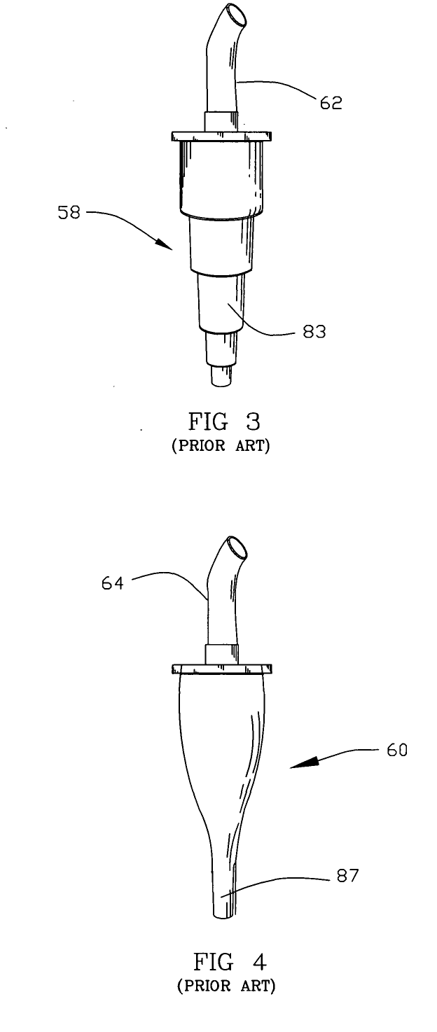

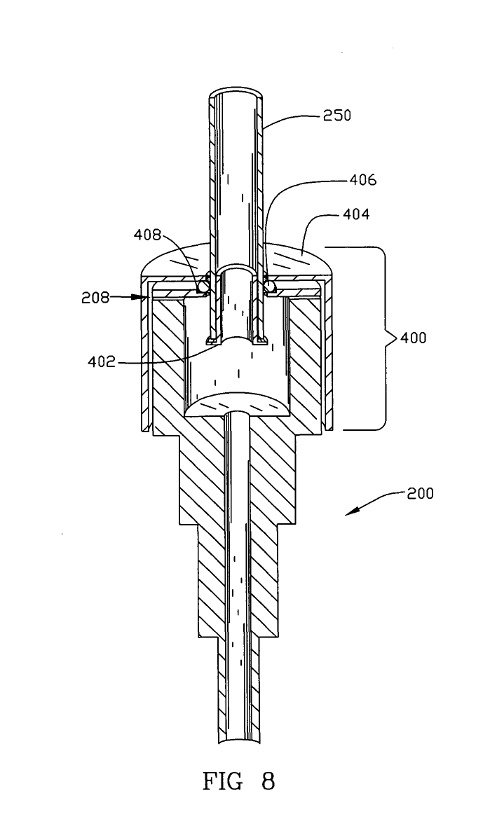

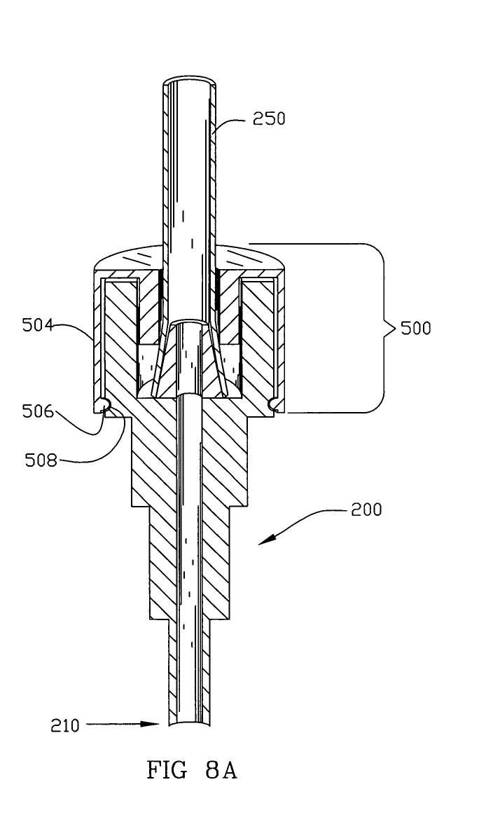

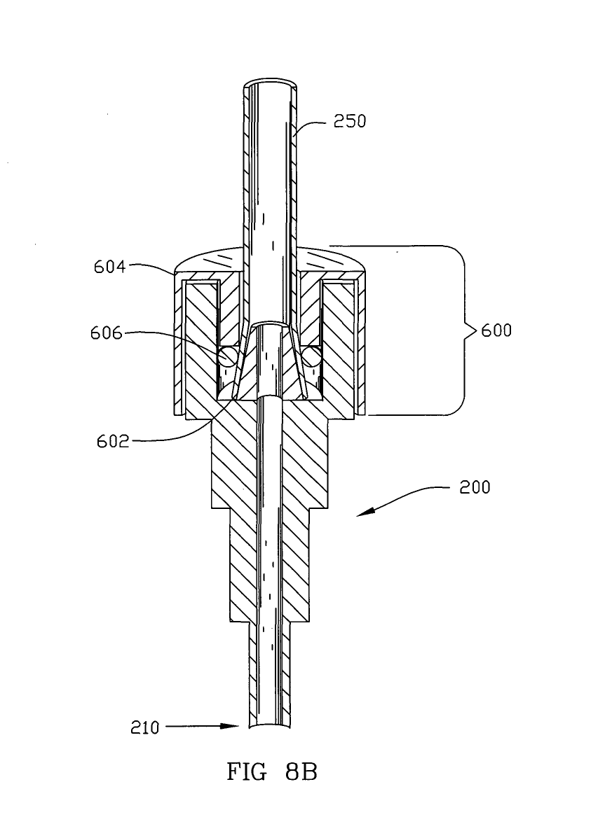

In the past, connectors such as those shown in FIGS. 3 and 4, used with feeding bags and enteral feeding devices were permanently fixed to the feeding sets or bags because they were not intended for multiple use nor were they intended for attachment at the time or point of use to tubes, feed bags, feeding sets, or the like. Advantageously, the connectors of the present invention are not permanently attached (e.g. sonically bonded, glued, etc.) to the tube or feeding set they are intended to be used therewith. That is, one or more embodiments of the present invention can have a connection mechanism (such as those shown as 300, 400, 500 and 600 in FIGS. 5, 8, 8A and 8B, respectively) at the first end 208 (FIGS. 5, 5A and 7-8B) of the body 204 (FIGS. 5, 5A and 7-8B) which may be used to releasably attach the connector 200 (FIGS. 5, 5A and 7-8B) to a tube 250 (FIGS. 6A and 8-8B). It will be appreciated that while a few suitable connection mechanisms are specifically identified, described and/or illustrated herein, any suitable connection mechanism is contemplated by the present invention. Examples of suitable connection mechanisms include, but are not limited to, slip connect, screw connect, slip fit, friction fit, grommets, compression fittings, compression sleeves and the like.

Referring now to FIGS. 6 and 7, there is shown an exemplary configuration of a second member, and more specifically, in this embodiment, a ferrule 70, which is suitable for receiving a connector of the present invention. As shown, this embodiment includes a slot 77 which extends through the wall 71 of the ferrule. As illustrated the slot has a first portion or leg 77a and a second portion or leg 77b. The slot 77 is adapted to receive a protrusion 202 (FIGS. 5, 5A and 7) on a connector 200 (FIGS. 5, 5A and 7) inserted into ferrule 70 (FIGS. 6 and 7). It is contemplated that the slot 77 may extend through entire width of the wall 71 of the ferrule 70, however, it is also contemplated that the slot may only extend through a portion of the wall 71 of the ferrule 70 (e.g. as to form a recess in the wall). Also, while shown between end surface 73 and rib 74a in FIG. 6, slot 77 may be in any suitable position in ferrule 70. That is, for example, at least a portion of the slot may extend through at least a portion of the end surface 73 (FIG. 6) of the ferrule; however, depending on the size of the connector and the location of the protrusion 202 (FIGS. 5, 5A and 7) thereon, the slot 77 (FIGS. 6 and 7) in the ferrule 70 (FIGS. 6 and 7) may be located closer to the end 75 (FIGS. 6 and 7) of the ferrule opposite end surface 73, and the slot need not extend into or through end surface 73. Depending on the size of the slot 77 (FIGS. 6 and 7) and the extent to which it extends into or through the wall 71 (FIGS. 6 and 7) of the ferrule 70 (FIGS. 6 and 7), a suitable connector may be selected. Alternatively, a suitable second member may be selected based on the dimensions of a connector that is selected for use.

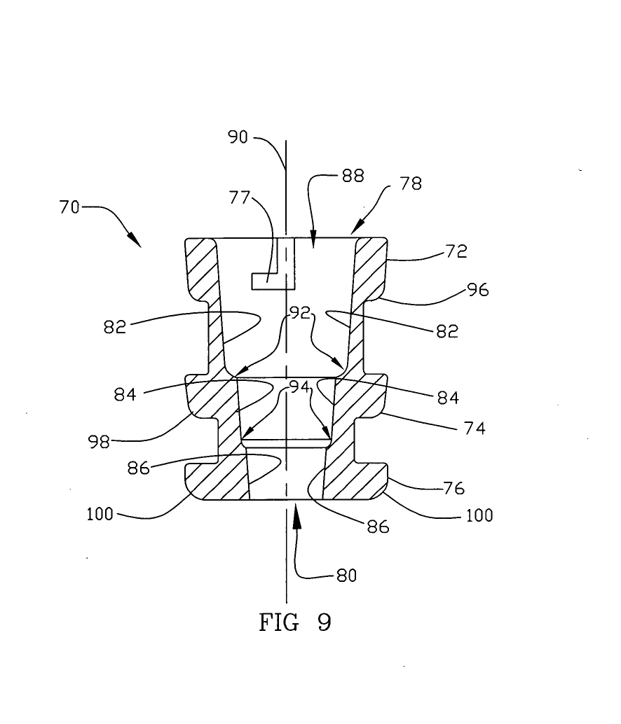

The illustrative drawing of FIG. 9, shows a cross-sectional view of the ferrule 70 of FIG. 6. First, second and third interior wall regions exemplarily shown as 82, 84 and 86 define a conduit 88 extending between the inlet opening 78 and the outlet opening 80. The respective first, second and third interior wall regions 82, 84 and 86 each desirably have substantially cylindrical contours and are aligned along a central axis 90 of the conduit 88.

As explained more fully below, the first, second and third interior wall regions 82, 84 and 86 may define three separate surfaces which may be taper lock surfaces. Each of these interior wall regions can be sized and contoured to conform to the shape of a different portion of a connector or to multiple connectors so as to enable the use of ferrule 70 with a variety of connectors. For example, the first interior wall region 82 can be sized and contoured to conform to the shape of a portion of a connector such as that shown as 58 in FIG. 3 or 60 in FIG. 4 or 200 in FIGS. 5, 5A and 7-8B. The second interior wall region 84 (FIG. 9), for example, can be sized and contoured to conform to the shape of another portion of the connectors in FIGS. 3-5A and 7-8B or another connector altogether. Moreover, the third interior wall region 86 (FIG. 9), for example, can be sized and contoured to conform to the shape of yet another connector (not shown) with a narrower body shape or another portion of the connectors discussed above. It will be appreciated that the interior wall regions of FIG. 9 are drawn to a different scale than the connectors of FIGS. 3-5A and 7-8B.

A taper lock may be caused by a frictional engagement force that results when a connector 200 (FIGS. 5, 5A and 7-8B) becomes lodged within the ferrule 70 (FIGS. 6 and 7) because of, for example, tapered surfaces 82, 84, 86, 92, 94 (FIG. 6) or 216, 222a, 224a, 226a (FIG. 5A) which come in contact with another surface which is also desirably tapered.

Thus, for example, when a connector such as that shown as 200 in FIGS. 5, 5A and 7-8B is inserted into the inlet opening 78 (FIG. 9) of the ferrule 70 (FIGS. 6 and 7), it can become lodged against the first interior wall region 82 (FIG. 9), desirably forming a taper lock with it. Alternatively, for example, connector 58 (FIG. 3) could become lodged against a shoulder of the ferrule such as 92 or 94 (FIG. 9). Likewise, when a connector having appropriate dimensions is inserted into the inlet opening 78 of the ferrule 70, it can become lodged against one or more of the other interior wall regions (e.g. 84 or 86), forming a taper lock or the like therewith. In accordance with the present invention, it will be appreciated that the creation of a taper lock or the like between the ferrule 70 and a connector desirably should not prevent the rotation or movement of connector relative to the ferrule 70 so as to enable the interlocking discussed herein. As discussed below, the present invention also contemplates instances in which a taper lock or other seal between the ferrule 70 and one or more wall regions of a connector is not made.

A more detailed description of the desired sizing and contour of one embodiment of the ferrule 70 of FIG. 9 follows. The first interior wall region 82 is shown with walls 82, 84 and 86 that are inclined relative to the central axis 90 so as to define a generally conical shape in which the diameter of a first segment of the conduit 88 defined by the first interior wall region 82 decreases with increasing distance from the inlet opening 78. A first interior annular shoulder 92 demarcates the end of the first interior wall region 82 of the ferrule of FIG. 9.

As will be appreciated, the interior walls of a ferrule may engage one or more of the regions of the body 204 (FIGS. 5, 5A and 7-8B) of a connector 200 (FIGS. 5, 5A and 7-8B) of the present invention so as to assist in the connection and/or retention of the components relative to each other and/or to reduce or minimize fluid leaks between the components.

A second segment of the conduit 88 is shown in FIG. 9 as being defined by the second interior wall region 84 which also is substantially conical in shape. Like the first interior wall region 82, the walls of the second interior wall region 84 may be inclined relative to the central axis 90 such that the diameter of a second conduit segment decreases with increasing distance from the inlet opening 78. A second interior annular shoulder 94 demarcates the end of the second interior wall region 84 of the ferule of FIG. 9.

A third segment of the conduit 88 is shown in FIG. 9 as being defined by the third conical interior wall region 86. The walls of the third interior wall region 86 are shown inclined relative to the central axis 90 such that the diameter of the third conduit segment decreases with increasing distance from the inlet opening 78. In one embodiment, the dimensions of the third interior wall region 86 may be those of a luer so as to be adopted to engage a portion of a connector.

It will be understood that although the above described embodiments disclose smooth inner wall regions (e.g. 82, 84, 86 shown in FIG. 9), a taper-lock can be formed in which inner wall regions have contours formed in them such as ridges, steps, bumps and the like. Alternatively, the connector may be formed such that the outer surfaces (e.g. 214 in FIG. 5) have contours formed in them such as ridges, steps, bumps and the like.

As noted and discussed in more detail herein, a connector 200 (FIGS. 5, 5A and 7-8B) and ferrule 70 (FIGS. 6 and 7) may create a lock or seal (e.g. a taper-lock) between one another; however, as discussed in more detail below, the ferrule 70 also has at least one slot 77 (FIGS. 6 and 7) therein which is capable of receiving a protrusion 202 (FIGS. 5, 5A and 7) on a connector 200 such as that shown in FIGS. 5, 5A and 7. It will be appreciated that while the term slot is used throughout this disclosure for ease of reading and understanding, and while not intending to be limited thereby, the term slot is intended to also include groove, channel, opening, recess, aperture and the like. It will also be appreciated that while the term protrusion is used throughout this disclosure for ease of reading and understanding, and while not intending to be limited thereby, the term slot is intended to also include, any suitable guide, flange, extension, prong, or the like also may be used.

Although illustrated in FIGS. 6 and 7 in a generally “L” shape, slot 77 of the ferrule 70 may be of any suitable shape and size. That is, for example, the second portion 77b of an “L-shaped slot” may be inclined or declined relative to the central axis 90 (FIG. 9) of the ferrule 70 (FIGS. 6 and 7) or the slot may be at least partially curved, T-shaped, U-shaped or the like. It is also contemplated that the slot 77 may have in at one least portion thereof a non-smooth surface or other mechanism (not shown) so as to enhance the ability of the ferrule 70 (FIGS. 6 and 7) to retain its position with a connector 200 (FIGS. 5, 5A and 7-8B) so as to minimize or reduce inadvertent or undesired separation of the components.

Although desired that the connector of the present invention be used with a ferrule having a slot, it is contemplated that a connector in accordance with the present invention may be used in conjunction with either a ferrule 70 (FIGS. 6 and 7) with a slot 77 (FIGS. 6 and 7) as discussed above or with a ferrule lacking a slot capable of receiving the protrusion of the connector. When the connector 200 (FIGS. 5, 5A and 7-8B) of the present invention is used in conjunction with a ferrule having a slot which is capable of receiving the protrusion 202 (FIGS. 5, 5A and 7) on the connector 200 and acting in conjunction with the projection 202 to retain or assist in maintaining the positioning of the connector 200 (FIGS. 5, 5A and 7) relative to the ferrule 70 (FIGS. 6 and 7), the protrusion 202 (FIGS. 5, 5A and 7) should be aligned with the slot 77 (FIGS. 6 and 7) to allow the connector 200 (FIGS. 5, 5A and 7) to be inserted into the ferrule 70 as illustrated in FIG. 7. The ferrule 70 (FIGS. 6 and 7), and more specifically the slot 77 (FIGS. 6 and 7) therein, is desirably designed such that a protrusion 202 (FIGS. 5, 5A and 7) may be closely received within a first portion or leg 77a (FIGS. 6 and 7) of the slot 77 (FIGS. 6 and 7). Then, the connector 200 (FIGS. 5, 5A and 7) and protrusion 202 (FIGS. 5, 5A and 7) may be rotated relative to the ferrule 70 (FIGS. 6 and 7) so as to be received within an intersecting second portion or leg 77b (FIGS. 6 and 7) of slot 77 (FIGS. 6 and 7) in a manner to releasably interlock the connector 200 (FIGS. 5, 5A and 7) with the ferrule 70 (FIGS. 6 and 7). Such an interlocking connection can provide a way to retain the position of a connector relative to a ferrule, even in those instances where a taper lock or other connection or seal between the outer surface of the connector and the ferrule (as discussed above) is not present or achieved.

It will be appreciated that in any embodiment, but especially those where a taper lock or the like is not created between the connector and the second member, the connector may include a sealing member or the like (not shown) desirably made of an elastomeric material or the like, which is capable of creating or forming a seal, gasketing effect, or the like with the second member the connector is intended to be used with. It will be appreciated that the sealing member may be located between the first end 208 (FIGS. 5, 5A and 7-8B) and the second end 210 (FIGS. 5, 5A and 7-8B) of the body 204 (FIGS. 5, 5A and 7-8B) of the connector 200 (FIGS. 5, 5A and 7-8B), but is desirably located closer to the first end 208 of the body 204 than the protrusion 202 (FIGS. 5, 5A and 7). Exemplary material the sealing member includes, for example, an elastomeric material or the like, and an exemplary embodiment may take the form of an o-ring, gasket or the like. Such a seal or the like can act to reduce, minimize or prevent leakage from the ferrule when the connector is inserted therein.

In the embodiment shown in FIG. 7, when it is desired to disconnect the connector 200 from the ferrule 70, the connector 200 and ferrule 70 should be rotated relative to one another such that the protrusion 202 is aligned with first portion or leg 77a of slot 77 of the ferrule 70. Once the protrusion is aligned with the leg 77a of slot 77, the connector 200 may be withdrawn from the ferrule 70 when sufficient force in the proper direction is applied.

Thus, the connectors 200 (FIGS. 5, 5A and 7-8B) in accordance with the present embodiments of the invention advantageously can be used to ensure that insertion, interconnection and removal from an end portion of a feeding tube or the like is facilitated without the exertion of undue force and without resulting in unwanted stretching of the end portion. Furthermore, the outer surface of connectors in accordance with the present invention can be sized and contoured to precisely conform to the shape of a number of different ferrule portions, however, as indicated above, the creation of a taper lock or the like between the connector and the ferrule while desired is not necessary. Further still, the connectors of the present invention advantageously can reduce or minimize undesired disconnections or separations from a second member. Such disconnections or separations may be avoided even in those instances in which a taper lock or the like is not achieved as was required with previous ferrules and connectors.

As indicated above, another advantage of the connectors of the present invention is the connection mechanisms. The connection mechanisms (such as 300, 400, and 500 shown in FIGS. 5, 8 and 8A, respectively) allow a connector 200 (FIGS. 5, 5A and 7-8B) which may have been selected for use because of its size and/or shape to be releasably connected with a tube 250 which may, for example, be part of a feeding set thereby providing for the ability to make a secure connection between the connector and the second member where such a connection might not otherwise have been obtainable. That is, for example, if a feeding tube, such as MIC* Gastrostomy Feeding Tube (Product # 100-16) (available from Ballard Medical Products, a subsidiary of the assignee of the present invention), which is designed so as to be able to satisfactorily receive a variety of connectors is desired for use with a feeding set which might not be designed for use therewith because of the size or shape of the connector affixed to the tube of the feeding set, the present invention enables a user to remove the original connector (e.g., cut it off) and then substitute a connector in accordance with the present invention onto the feeding set. The connection mechanism 300 (FIGS. 5 and 6A) of the present invention allows the tube 250 (FIGS. 6A and 8-8B) to be connected to the connector 200 (FIGS. 5, 5A and 7-8B) so as to create an association which desirably minimizes or avoids leaks between the connector and the tube. Further, when the feeding bag is empty, the user may disconnect the connector 200 from the tube 250 (FIGS. 6A and 8-8B) so as to make the connector available for future use with another feeding set. Thus, use of an undesirable or less desirable feed set or bag may be avoided as the present invention provides for use of a connector which is suitable for use with a feeding tube or catheter desired by the patient or clinician, even if the original connector on the desired feed set was not generally compatible with the desired feeding tube or catheter.

As above a variety of suitable connection mechanisms are contemplated by the present invention. For instance, one exemplary embodiment is shown in FIGS. 5 and 6A. As illustrated, the connection mechanism 300 includes a first member, and more specifically a compression fitting or sleeve 302 and a cap 304. In use the cap 304 desirably fits over the compression fitting 302 but each has a bore 306, 307 respectively as shown in FIGS. 5 and 6A. The compression fitting 302 and cap 304 work together to so as to allow the releasable connection of a tube 250 (FIGS. 6A and 8-8B). For example, when the compression fitting 302 and cap 304 are in the position illustrated in FIG. 7 a tube 250 (FIGS. 6A and 8-8B) may be inserted into bores 306 and 307. Once inserted into bores 306 and 307, the compression fitting 302 and cap 304 may work in conjunction with the inner surface 212 (FIG. 5) of the connector to retain the tube 250 (FIGS. 6A and 8-8B) therein. That is, in one embodiment, as pressure is applied to the tube 250 (FIGS. 6A and 8-8B) in a direction opposite that of inserting the tube into the connector, compression fitting 302 and cap 304 may try to move in said direction; however, as such movement occurs, sufficient pressure against the side of the tube 250 will be applied by one or more components of the connection mechanism 300 so as to prevent the removal of the tube 250 from the connection mechanism 300. When it is desired, the tube 250 may be removed from the compression fitting 302 and cap 304 by, for example, holding the connector mechanism 300 in a position such as that shown in FIG. 7 such that the compression fitting 302 and/or cap 304 (FIGS. 5 and 6A) are not applying the requisite pressure against the tube 250 (FIGS. 6A and 8-8B) to prevent the removal of the tube 250.

Still other variations of the connector mechanism discussed above are contemplated. For example, the cap 304 (FIGS. 5A and 6A) is not required in all embodiments having a compression fitting 302. That is, compression fitting 302 may act in conjunction with the connector 200 to retain the tube 250. Alternatively, rather than the compression fitting 302 and cap 304 being separate pieces they can be integrally formed and/or connected.

Another exemplary embodiment of a connection mechanism is shown in FIG. 8. In that embodiment, the connection mechanism 400 includes a grommet 402 and cap assembly. Although the cap assembly is shown as including a cap 404, an o-ring 406 and an o-ring seat 408, the o-ring 406 and o-ring seat 408 may only be present in some embodiments. In use, the tube 250 is desirably inserted or pushed onto the grommet 402 and the cap 404 may be positioned such that the tube 250 is held in place. Desirably the cap 404 may be positioned in a tightened or retaining position so as to apply pressure to the outside of the tube 250 and in such a manner as to grip or sandwich the tube 250 between the cap 404 and the grommet 402. The application of pressure may be direct or indirect. That is, for example, the cap 404 may contact the tube 250 directly (not shown) so as to retain the tube within the connector or the cap 404 may contact the o-ring 406 which may be seated in a groove or o-ring seat 408 such that when the cap 404 is put into in a tightened or retaining position the cap 404 may cause the o-ring 406 to contact or apply more pressure to the tube 250 so as to retain the tube within the connector.

All suitable ways for tightening and/or keeping the cap 402 in a tightened or retaining position are contemplated by the present invention. Exemplary ways include, but are not limited to, a threaded cap 404 such as that shown in FIG. 8 which may be screwed or tightened onto the end 208 of a connector 200 by turning the cap 404. Although a separate cap is contemplated, the cap is desirably such that while it may be loosened so as to allow release of the tube 250, the cap 404 may not be completely removed from the connector. Alternatively, as shown in FIG. 8A, the cap 504 may have a boss 506 and the connector 200 a groove or detent 508 such that when the cap 504 is tightened about the tube 250 so as to retain it, the boss 506 and detent 508 are in communication so as to maintain the position of the cap 504 relative to the connector 200.

Yet another exemplary embodiment of the connection mechanism is shown in FIG. 8B. In FIG. 8B a tube 250 is placed about a grommet (not shown) or molded nipple 602 which may be part of the connector 200. The nipple 602 may have a flared shape with the cross-section of the nipple 602 increasing in dimension as the distance to the second end 210 of the connector 200 decreases. As the tube 250 is pushed over the nipple 602, the tube 250 may flare out and may be held in place thereby. Desirably, however a cap 604 will also be present which may apply pressure directly or indirectly (e.g. via an o-ring or the like) to the tube 250 so as to retain its position relative to the connector 200.

As will be appreciated, numerous suitable ways of retaining a tube in releasable communication with a connection mechanism and/or connector exist. All such suitable ways are contemplated by the present invention.

While the invention has been described in detail with respect to specific embodiments thereof, those skilled in the art, upon obtaining an understanding of the invention, may readily conceive of alterations to, variations of, and equivalents to the described embodiments and the processes for making them. It is intended that the present invention include such modifications and variations as come within the scope of the appended claims and their equivalents.

1. A connector adapted for use with a feeding set formed from flexible material comprising:
a body with a conduit therethrough, the body having a first end, a second end, an inner surface and an outer surface; and
a connecting mechanism for releasably securing a tube to the first end of the connector.

2. The connector of claim 1 wherein the outer surface has at least two regions having different diameters; and
the outer surface of the body has at least one bevel, wherein the bevel connects two regions along the surface of the body having different diameters.

3. The connector of claim 1 wherein the body has multiple tapers along the outer surface.

4. The connector of claim 1 further including a protrusion extending from the outer surface of the body, the protrusion configured to be received in a second member so as to allow the connector and the second member to be releasably interconnected.

5. The connector of claim 1 wherein the connecting mechanism is selected from the group consisting of compression fitting, compression sleeve, slip-fit, slip connect, screw connect, slip fit, and grommets.

6. The connector of claim 1 wherein the connecting mechanism comprises a compression fitting.

7. The connector of claim 1 wherein the connecting mechanism comprises a cap.

8. The connector of claim 1 wherein the connecting mechanism comprises a compression fitting and a cap.

9. The connector of claim 1 wherein the connecting mechanism comprises a cap and a grommet.

10. The connector of claim 7 wherein the connecting mechanism further comprises an o-ring.

11. The connector of claim 1 wherein the connecting mechanism comprises a cap and a nipple.

12. The connecting mechanism of claim 9 wherein the cap is at least partially threaded.

13. A universal connector adapted for use with a medical device comprising:
a body with a conduit therethrough, the body having a first end and a second end, and an inner surface and an outer surface; and
a mechanism for releasably securing a tube to the first end of the connector;
wherein the outer surface having at least two regions having different diameters; and the outer surface of the body has at least one bevel, wherein the bevel connects two regions along the surface of the body having different diameters.

14. The universal connector of claim 13 further comprising a protrusion extending from the outer surface of the body, the protrusion configured to be received in a second member so as to allow the connector and the second member to be releasably interconnected.

15. The universal connector of claim 13 adapted for use with a feeding tube.

16. The universal connector of claim 13 adapted for use with a feeding set.

17. The universal connector of claim 13 having a flexible tube releasably secured thereto.

18. A method of using a universal connector adapted for use with a medical device, the method comprising:
providing a connector comprising:
a body with a conduit therethrough, the body having a first end, a second end, an inner surface and an outer surface; and
a mechanism for releasably securing a tube to the first end of the connector;
wherein the outer surface having at least two regions having different diameters; and the outer surface of the body has at least one bevel, wherein the bevel connects two regions along the surface of the body having different diameters;
providing a tube having two ends; and
inserting one end of the tube into the mechanism.

19. The method of claim 18 wherein the connector further includes a protrusion extending from the outer surface of the body, the protrusion configured to be received in a second member so as to allow the connector and the second member to be releasably interconnected.

20. The method of claim 18 wherein the tube is part of a feeding set.

21. The method of claim 18 further comprising releasably securing the mechanism so as to retain the tube.

22. The method of claim 18 wherein the tube may be released from the mechanism.

23. The method of claim 18 wherein the mechanism for releasably securing the tube comprises a cap and a grommet.

24. The method of claim 18 wherein the mechanism for releasably securing the tube comprises a compression fitting.

25. The connector of claim 18 wherein the connecting mechanism comprises a cap and a nipple.
